(19) United States
(12) Patent Application Publication
(10) Pub. No.: US 2022/0177517 A1
(43) Pub. Date: Jun. 9, 2022

(54) PROCESS FOR THE PURIFICATION OF RECOMBINANT POLYPEPTIDES

(71) Applicant: GlaxoSmithKline Intellectual Property Development Limited, Brentford Middlesex (GB)

(72) Inventors: Daniel GIBSON, Stevenage (GB); Richard KUCIA-TRAN, Stevenage (GB); Nehal PATEL, Stevenage (GB); Trevor WILEY, King of Prussia, PA (US)

(21) Appl. No.: 17/600,276

(22) PCT Filed: Apr. 3, 2020
(86) PCT No.: PCT/EP2020/059589

§ 371 Date: Sep. 30, 2021

Related U.S. Application Data
(60) Provisional application No. 62/991,700, filed on Mar. 19, 2020.

(30) Foreign Application Priority Data
Apr. 4, 2019 (GB) 1904741.4

(51) Int. Cl. C07K 1/22; C07K 16/06; C07K 1/16; C07K 1/18; C07K 1/20

(52) U.S. Cl. CPC C07K 1/22; C07K 16/065; C07K 2317/31; C07K 1/18; C07K 1/20; C07K 1/165

(57) ABSTRACT

The present invention is directed to a novel process for purifying a recombinant polypeptide from a solution comprising one or more impurities, wherein the process is a chromatography process which uses saccharin. The present invention also provides the use of saccharin in a process for purifying a recombinant polypeptide from a solution comprising one or more impurities, wherein the process is a chromatography process. The invention further provides a wash buffer for purifying using chromatography a recombinant polypeptide from a solution comprising one or more impurities, wherein the wash buffer comprises saccharin.

### FIELD OF THE INVENTION

The present invention is directed to a novel process for purifying a recombinant polypeptide from a solution comprising one or more impurities, wherein the process is a chromatography process which uses saccharin. The present invention also provides the use of saccharin in a process for purifying a recombinant polypeptide from a solution comprising one or more impurities, wherein the process is a chromatography process. The invention further provides a wash buffer for purifying a recombinant polypeptide from a solution comprising one or more impurities, wherein the wash buffer comprises saccharin.

### BACKGROUND TO THE INVENTION

Recombinant polypeptides, such as antibodies and other proteins, are used for the therapeutic treatment of a wide range of diseases. The biopharmaceutical manufacture of these complex recombinant polypeptides typically requires the use of a biological host system, which through genetic engineering, is capable of expressing the product in a suitably active form. Expression of recombinant polypeptides generally involves culturing prokaryotic or eukaryotic host cells under appropriate conditions. Once the recombinant polypeptide is expressed, intact host cells and cell debris can be separated from the cell culture media to provide a clarified unprocessed bulk (CUB) or clarified cell culture fluid (CCCF), which includes the recombinant polypeptide and other impurities.

Recombinant polypeptides produced by biopharmaceutical manufacturing processes are typically associated with multiple undesirable impurities, including, but not limited to: host cell proteins (HCPs), DNA, viruses, high- and low-molecular weight species, and unwanted product and process variants, which can be difficult to remove and have the potential to significantly reduce the safety and efficacy of the biopharmaceuticals manufactured. The levels of the impurities therefore must be critically controlled to comply with regulatory guidelines, and the added complexity of contaminants with different physicochemical properties makes identification, quantification, and removal of them and their residual amounts even more challenging, particularly in the presence of large concentrations of the desired recombinant polypeptide product.

Multiple orthogonal processes of purification are often required during downstream biopharmaceutical processing to produce a sufficiently pure recombinant polypeptide.

There exists a need to provide an improved process for purifying recombinant polypeptides.

### SUMMARY OF THE INVENTION

The present invention provides a process for purifying a recombinant polypeptide from a solution comprising one or more impurities, wherein the process comprises the addition of saccharin.

The present invention provides a process for purifying a recombinant polypeptide from a solution comprising one or more impurities, wherein the solution is a cell culture feedstream and the process comprises the addition of saccharin.

The present invention provides a process for purifying a recombinant polypeptide from a solution comprising one or more impurities, wherein the process is a chromatography process which comprises the addition of saccharin.

The present invention further provides a wash or load buffer for purifying using chromatography a recombinant polypeptide from a solution comprising one or more impurities, wherein the wash or load buffer comprises saccharin. The present invention further provides a cell culture feedstream comprising a recombinant polypeptide and one or more impurities, wherein the feedstream is a solution comprising saccharin.

### BRIEF DESCRIPTION OF FIGURES

FIG. 1: The HCP impurity levels (ppm) were measured in six Protein A chromatography eluates containing recombinant polypeptide. HCP levels measured in five of the eluates contained either 0, 50, 280, 500, or 930 mM saccharin in the initial CUB (CCCF) Protein A load, whereas the sixth eluate contained 0 mM saccharin and was eluted under control conditions. Increasing the saccharin concentration in the Protein A load was shown to increase HCP clearance. The addition of ≥280 mM of saccharin to the load was enough to give greater HCP clearance when compared to the Caprylate wash. However, the recombinant polypeptide monomer levels measured for each of the six Protein A eluates were found to be very similar, all six contained 98.6±0.3% monomer.

FIG. 2: A MABSELECT SURE (MSS) chromatogram of a recovered antibody known as mAb2 after Caprylate wash under control conditions (Table 2) was taken. The antibody mAb2 eluted at about 120 ml. The antibody mAb2 monomer purity was found to be 93.6%, and HCP content 4517.87 ppm.

FIG. 3: An MSS chromatogram of the same recovered antibody mAb2 of FIG. 2 was taken, but this time after using an arginine wash, wherein the wash buffer contained 1.1 M arginine in the Caprylate wash (Table 2). The antibody mAb2 monomer purity was found to be 96.1%, and HCP content 357.20 ppm.

FIG. 4: An MSS chromatogram of the same recovered antibody mAb2 of FIGS. 2 and 3 was taken, but this time after a saccharin wash; wherein the wash buffer contained 0.5 M saccharin in the equilibration buffer (Table 2). The recombinant polypeptide monomer purity was found to be 97.3%, and HCP content 352.20 ppm.

### DETAILED DESCRIPTION OF THE INVENTION

The present invention provides a process for purifying a recombinant polypeptide from a solution comprising one or more impurities, wherein the process comprises the addition of saccharin.

The solution may be a cell culture feedstream. This may be a harvested feedstream or a continuous feedstream. The solution may be a continuous feedstream from a bioreactor. The solution may be a Clarified Unprocessed Bulk (CUB) (or clarified cell culture harvest/supernatant/fermentation/ fluid). The CUB is also known as a cell culture supernatant with any cells and/or cellular debris removed by clarification. Host cells and cell debris can be separated from the cell culture media by clarification, for example via sedimentation, centrifugation and/or filtration. The solution may be a lysed preparation of cells expressing the recombinant polypeptide (e.g. a lysate). The solution may be a clarified cell culture fluid (CCCF). Clarified cell culture fluid (CCCF) is equivalent to Clarified Unprocessed Bulk (CUB) and both terms can be used interchangeably.

The bioreactor may be a production bioreactor, or n-1 bioreactor, or n-2 bioreactor. The bioreactor may operate in perfusion mode or fed-batch or batch or combinations thereof. The bioreactor may be at a scale of 500 litres, 1000 litres, 2000 litres, 3000 litres, 4000 litres, or 5000 litres or greater. The bioreactor may be at a scale of 10,000 litres, 15,000 litres, 20,000 litres, 25,000 litres, or 30,000 litres or greater. The bioreactor may be single use or fixed. The bioreactor may be suitable for recombinant polypeptide production in a mammalian host cell. The mammalian host cell may be selected from: CHO, NS0, Sp2/0, COS, K562, BHK, PER.C6, and/or HEK cells. In one aspect, the host cell is a Chinese Hamster Ovary cell line (CHO).

The solution may comprise a buffer. For example the solution may comprise a load buffer, an equilibration buffer, a wash buffer, and/or an elution buffer. The solution may comprise the eluate from a chromatography step. The solution comprising the recombinant polypeptide and one or more impurities and saccharin may be purified by subsequent purification steps. These steps may or may not comprise the addition of saccharin. These steps may or may not comprise chromatography steps. The resulting purified solution may be formulated for therapeutic use. The purification steps may not comprise sodium chloride.

The present invention provides a process for purifying a recombinant polypeptide, wherein the process is a chromatography process which uses saccharin. For example, the recombinant polypeptide is purified from a solution comprising one or more impurities.

The present invention also provides the use of saccharin in a process for purifying a recombinant polypeptide, wherein the process is a chromatography process. For example, the recombinant polypeptide is purified from a solution comprising one or more impurities.

The present invention further provides a wash buffer for purifying using chromatography a recombinant polypeptide, wherein the wash buffer comprises saccharin. For example, the recombinant polypeptide is purified from a solution comprising one or more impurities.

Herein described is the use of saccharin in a process for purifying a recombinant polypeptide from a solution comprising one or more impurities. Herein described is the use of saccharin in a process for purifying a recombinant polypeptide from a solution comprising one or more impurities, wherein the process is a chromatography process.

In one embodiment, the process comprises: (a) a loading step; (b) a washing step; and/or (c) an eluting step. In one embodiment, the purified recombinant polypeptide is (i) optionally further purified and (ii) formulated for therapeutic use. In a further aspect, the purified recombinant polypeptide is recovered from the eluate of step (c), and optionally formulated.

In one embodiment, the chromatography process comprises one or more chromatography methods. For example, the one or more chromatography methods comprise affinity chromatography; ion exchange chromatography; anion exchange chromatography; cation exchange chromatography; hydrophobic interaction chromatography (HIC); mixed mode chromatography (MMC); and/or ceramic hydroxyapatite chromatography. In one embodiment, the one or more chromatography methods comprise affinity chromatography. In one embodiment, the one or more chromatography methods comprise Protein A affinity chromatography.

In one embodiment, the process comprises (i) any one or a combination of affinity chromatography; ion exchange chromatography; anion exchange chromatography; cation exchange chromatography; hydrophobic interaction chromatography (HIC); mixed mode chromatography (MMC); and/or ceramic hydroxyapatite chromatography; and (ii) any one or a combination of (a) a loading step, (b) a washing step and/or (c) an eluting step.

In one embodiment, the process used is liquid chromatography. For example, the process used is: affinity chromatography; ion exchange chromatography; anion or cation exchange chromatography; gel-permeation or gel-filtration chromatography; dye-ligand chromatography;

hydrophobic interaction chromatography (HIC); mixed mode chromatography (MMC); or ceramic hydroxyapatite chromatography. In one aspect, the process is affinity chromatography.

The chromatography process is carried out using a chromatography support and a mobile phase; wherein the chromatography support is either aqueous or non-aqueous. In one embodiment, the non-aqueous phase comprises: agarose, sepharose, glass, silica, polystyrene, collodion charcoal, sand, polymethacrylate, cross-linked poly(styrene-divinylbenzene), agarose with dextran surface extender, or any other suitable material. For example, the non-aqueous phase is MABSELECT SURE resin. In a further aspect, the non-aqueous phase is linked to an affinity ligand, for example Protein: A; G; L; or A/G. In one aspect, the affinity ligand is Protein A. In one aspect the non-aqueous phase is cation exchange chromatography.

The affinity ligand may be from a native source or synthetic, or a synthetic variant thereof. In one embodiment, the protein A used is from a native source or it is synthetic, or it is a synthetic variant thereof which has the ability to bind polypeptides with a C_{H}2/C_{H}3 region. Protein A can bind to the Fc region and can also bind to the variable region of the heavy chain (VH3), the affinity of which is strengthened in the absence of an Fc region. Protein L can bind to the variable region of the light chain. Protein G can bind to the Fc region and can also bind to the variable region (Fab). Thus, affinity chromatography using one or more of Protein A, Protein L, or Protein G can be used to purify a number of different antigen binding proteins such as IgG, scFv, dAb, Fab, diabody, nanobody, Fc-containing fusion protein, i.e. including those that do not contain Fc regions. The use of Protein A, Protein L, and Protein G to purify such antigen binding proteins is known and is routine in the art.

In one embodiment, the chromatography process comprises: a loading step; a washing step; and/or an eluting step.

In one embodiment, the chromatography process comprises a loading step, wherein the loading step comprises the addition of saccharin.

In one embodiment, the chromatography process comprises a washing step, wherein the washing step comprises the addition of saccharin.

In one embodiment, the chromatography process comprises a loading step and/or a washing step, wherein the loading step and/or the washing step comprises the addition of saccharin.

In one embodiment, the chromatography process comprises an eluting step, wherein the eluting step does not comprise the addition of saccharin.

In a particular embodiment of the present invention, the saccharin used in the process is present in the: (a) loading step; (b) washing step; and/or (c) eluting step.

In one embodiment, the saccharin used in the process is in salt form. In one aspect, the saccharin is in the form of: sodium saccharin (also known as: o-sulfobenzimide sodium salt; 2-sulfobenzoic acid imide sodium salt; or 2,3-dihydro-3-oxobenzisosulfonazole sodium salt), for example saccharin sodium salt hydrate (also known as: 2,3-dihydro-3-oxobenzisosulfonazole hydrate), saccharin sodium salt dehydrate, or saccharin sodium salt dihydrate; saccharin calcium; saccharin hemicalcium salt; 2-sulfobenzoic acid ammonium salt; or aluminium saccharin salt. In one embodiment, the saccharin is in the form of 2-sulfobenzoic acid ammonium salt; saccharin sodium salt dihydrate; or saccharin sodium salt hydrate. In one aspect, the saccharin is in the form of saccharin sodium, for example saccharin sodium salt hydrate. In an alternative aspect, the saccharin used in the process may be in the form of N-(2-nitrophenylthio)saccharin. Sodium saccharin dihydrate is interchangeable with saccharin sodium salt dihydrate. Saccharin sodium hydrate or sodium saccharin hydrate are both interchangeable with saccharin sodium salt hydrate. 2-sulfobenzoic acid ammonium salt is interchangeable with 2-sulfobenzoic acid (saccharin) ammonium salt.

In a further embodiment, the saccharin concentration used in the process is about 0.001 to about 4 M; about 0.1 to about 3 M; about 0.1 to about 2 M; or about 0.1 to about 0.9 M. The saccharin concentration may be about 0.5 to about 1.5 M, or about 0.6 to about 1.5 M. The saccharin concentration may be about 0.7 to about 1.5 M, or about 0.75 to about 1.5M. The saccharin concentration may be about 0.5 to about 1.0 M, or about 0.6 to about 1.0 M. The saccharin concentration may be about 0.7 to about 1.0 M, or about 0.75 to about 1.0 M. The saccharin concentration may be about 0.6 to about 1.4 M, or about 0.7 to about 1.3 M, or about 0.8 to about 1.2M. In a particular aspect, the saccharin concentration is selected from about: 0.1 M, 0.15 M, 0.2 M, 0.25 M, 0.275 M, 0.3 M, 0.325 M, 0.35 M, 0.375 M, 0.4 M, 0.425 M, 0.45 M, 0.475 M, 0.5 M, 0.525 M, 0.55 M, 0.575 M, 0.6 M, 0.625 M, 0.65 M, 0.7 M, 0.725 M, 0.75 M, 0.8 M, 0.9 M, or 1 M. In one embodiment, the saccharin concentration is about 0.01 to about 4 M; about 0.01 to about 3 M; about 0.05 to about 3 M; about 0.05 to about 1 M; about 0.1 to about 3 M; about 0.1 to about 1 M; about 0.2 to about 3 M; about 0.2 to about 1.5 M; about 0.2 to about 1 M; about 0.2 to about 0.8 M; about 0.2 to about 0.6 M; about 0.3 to about 3 M; about 0.3 to about 1.5 M; about 0.3 to about 1 M; about 0.3 to about 0.8 M; or about 0.3 to about 0.5 M. In one aspect, the saccharin concentration is about 0.3 M to about 0.5 M. In an alternative aspect, the saccharin concentration used in the process is increased, for example from about 1 mM, 10 mM, 50 mM, 0.1 M, 0.3 M, 0.4 M, 0.5 M, 0.6 M, 0.7 M, 0.8 M, 0.9 M, or 1 M.

Saccharin may used in combination with one or more additives. For example, saccharin may be used in a purification process wherein the additive is used with saccharin at the same time, or subsequent to or before the addition of saccharin. Saccharin may be added to a process at the same time as the additive. Saccharin may be added to the solution at the same time as the additive. The additive may be an aliphatic carboxylate or salt thereof such as caproate, heptanoate, caprylate, decanoate, and dodecanoate. For example the additive is sodium caprylate. The additive may be arginine. The additive may be lysine. The additive may be sodium acetate. The additive may be sodium chloride. Saccharin may be used at the same time as caprylate. Saccharin may be used at the same time as arginine. Saccharin may be used at the same time as sodium acetate. Saccharin may be used at the same time as caprylate and sodium acetate. Saccharin may be used at the same time as sodium acetate and arginine. Saccharin may be used at the same time as caprylate and arginine. Saccharin may be used at the same time as caprylate, arginine and sodium acetate.

The solution comprising the recombinant polypeptide and one or more impurities may comprise one or a combination of saccharin, an aliphatic carboxylate or salt thereof, arginine, lysine, sodium acetate and/or sodium chloride. The solution comprising the recombinant polypeptide and one or more impurities may comprise one or a combination of saccharin, caprylate, sodium acetate and/or arginine. The solution comprising the recombinant polypeptide and one or more impurities may comprise saccharin and caprylate. The solution comprising the recombinant polypeptide and one or more impurities may comprise saccharin and arginine. The solution comprising the recombinant polypeptide and one or more impurities may comprise saccharin, sodium acetate and caprylate. The solution comprising the recombinant polypeptide and one or more impurities may comprise saccharin, sodium acetate and arginine. The solution comprising the recombinant polypeptide and one or more impurities may comprise saccharin, sodium acetate, caprylate and arginine.

The concentration of the aliphatic carboxylate or salt thereof may be about 1 to about 250 mM, or about 75 to about 250 mM, or about 100 to about 250 mM. For example, the concentration of sodium caprylate is about 100 mM to about 250 mM. For example, the concentration of sodium caprylate is about 100 mM. For example, the concentration of sodium caprylate is about about 250 mM.

The concentration of arginine may be about 0.1M to about 2M, or about 0.5M to about 1.5M, or about 0.75M to about 1.25M. For example, the concentration of arginine is about 1.1M.

The concentration of lysine may be about 0.5 M to about 1 M, for example about 0.75 M.

The concentration of sodium acetate may be about 0.1M to about 2M, or about 0.2M to about 1.5M, or about 0.2M to about 1.2M. For example, the concentration of sodium acetate is about 0.1M, about 0.3M or about 1M.

Saccharin is added to a solution comprising the recombinant polypeptide and one or more impurities. Saacharin may be added to the solution prior to any chromatography steps. The solution that is loaded onto a chromatography support may already comprise saccharin. For example the load may comprise: saccharin, the recombinant polypeptide and one or more impurities.

Saccharin may be added to a buffer. Saccharin may be added to a buffer for chromatography. For example the buffer may be a load buffer, an equilibration buffer, a wash buffer, and/or an elution buffer. The buffer may be at a pH of 5 to 9. The buffer may comprise one or more of: sodium acetate and acetic acid, phosphate-buffered saline (PBS), 2-(N-morpholino)ethanesulfonic acid (MES), tris base and acetic acid, and/or 3-(N-morpholino)propanesulfonic acid (MOPS).

In one embodiment, the solution comprising the recombinant polypeptide and one or more impurities is loaded onto the chromatography support in the loading step. In one aspect, the solution comprising the recombinant polypeptide and one or more impurities is CCCF. For example, the CCCF comprises saccharin. In one aspect, the load comprises saccharin. In another aspect, the load buffer comprises saccharin.

In one embodiment, the recombinant polypeptide is loaded onto the chromatography support in the presence of an equilibration buffer. For example, the solution comprises one or more impurities. In one aspect, the pH of the equilibration buffer is about 5.0-9.0, for example about 5.0-8.0. In one aspect, the equilibration buffer comprises tris base and acetic acid. In another aspect, the pH is about 7.5. In one aspect, the tris base concentration is about 55 mM and the acetic acid concentration is about 45 mM. And optionally, the equilibration buffer further comprises saccharin. In one aspect, the saccharin in the equilibration buffer is in the form of saccharin sodium salt hydrate. In yet a further aspect, the saccharin concentration in the equilibration buffer is about 0.5 to about 1.5 M, or about 0.3 to about 0.5 M.

In one embodiment, the washing step uses a wash buffer. Standard wash buffers are well known in the art, for example Holstein et al., (2015) BioProcess International, 13(2):56-62. In one aspect, the wash buffer comprises tris base; acetic acid; and/or sodium acetate. In one aspect, the wash buffer comprises tris base and acetic acid. In a further aspect, the wash buffer comprises an additive for example: an aliphatic carbon/late or salt thereof such as caproate, heptanoate, caprylate, decanoate, and dodecanoate; arginine; lysine; and/or sodium chloride. In one embodiment, the washing step which uses a wash buffer does not comprise sodium chloride. In an even further aspect, the additive concentration is about 1 to about 500 mM, or about 75 to about 300 mM. The additive concentration may be about 0.1 M to about 2 M.

In one embodiment, when the buffer in the wash buffer is tris base, the tris base concentration is about 55 mM, and when the buffer is acetic acid, the acetic acid concentration is about 45 mM acetic acid. In one aspect, when the buffer is sodium acetate, the sodium acetate concentration is about 300 mM to about 1 M. In a further aspect, when the additive is caprylate, the caprylate concentration is about 250 mM, or about 100 mM. In one aspect, the caprylate is sodium caprylate. In another aspect, when the additive is arginine, the arginine concentration is about 1 mM to about 2 M, such as about 1.1 M. In a yet another aspect, when the additive is lysine, the lysine concentration is about 0.5 M to about 1 M lysine, for example about 0.75 M lysine.

In one embodiment, the wash buffer comprises a saccharin concentration of 0.05-3 M, for example 0.05-1 M, or about 0.5 to about 1.5 M. In one aspect, the saccharin concentration in the wash buffer is 0.3 M. In another aspect, the wash buffer has a saccharin concentration of 0.5 M. In another aspect, the wash buffer has a saccharin concentration of about 1 M.

In one embodiment, the pH of the wash buffer used in the process is between about pH 5 to about pH 9, for example about pH 7 to about pH 9, for example from about pH 7.5 to about pH 8.5. In particular, the pH is about pH 7.5.

In one embodiment, the eluting step uses an elution buffer. In a particular aspect, the elution buffer is acidic, for example the pH is less than about 6.5. Suitable elution buffers are well known in the art. In a further aspect, the elution buffer comprises of: a salt; glycine; citric acid; sodium acetate; and/or acetic acid. In one embodiment, the elution buffer comprises sodium acetate and acetic acid. Suitable concentrations of the elution buffers, for example sodium acetate and acetic acid, used in the process will be apparent to one skilled in the art. For example, the sodium acetate concentration is 1.8 mM and acetic acid concentration 28.2 mM, and the pH of the elution buffer is 3.6.

In one embodiment, the eluting step does not comprise the addition of saccharin. In one embodiment, the elution buffer does not comprise saccharin. In an embodiment, saccharin is not used to displace the recombinant polypeptide from a chromatography support. In an embodiment, the process does not comprise displacement chromatography. In an embodiment, the process does not comprise displacement chromatography wherein saccharin is the displacer.

In one embodiment, the recombinant polypeptide used in the process is an antigen binding protein. In a further aspect, the antigen binding protein is selected from the group consisting of an antibody, antibody fragment, immunoglobulin single variable domain (dAb), mAbdAb, Fab, F(ab′)₂, Fv, disulphide linked Fv, scFv, closed conformation multispecific antibody, disulphide-linked scFv, diabody or a soluble receptor. In one aspect, the antigen binding protein is an antibody.

The term “antibody” is used herein in the broadest sense to refer to molecules with an immunoglobulin-like domain (for example IgG, IgM, IgA, IgD or IgE) and includes monoclonal, recombinant, polyclonal, chimeric, human, humanised, multispecific antibodies, including bispecific antibodies, and heteroconjugate antibodies; a single variable domain (e.g., a domain antibody (DAB)), antigen binding antibody fragments, Fab, F(ab′)₂, Fv, disulphide linked Fv, single chain Fv, disulphide-linked scFv, diabodies, TANDABS, etc. and modified versions of any of the foregoing (for a summary of alternative “antibody” formats see Holliger and Hudson, Nature Biotechnology, 2005, Vol 23, No. 9, 1126-1136).

The five classes of antibodies IgM, IgA, IgG, IgE and IgD are defined by distinct heavy chain amino acid sequences which are called μ, α, γ, ε and δ respectively, each heavy chain can pair with either a K or λ light chain. The majority of antibodies in the serum belong to the IgG class, there are four isotypes of human IgG, IgG1, IgG2, IgG3 and IgG4, the sequences of which differ mainly in their hinge region.

The term multi-specific antigen binding protein refers to antigen binding proteins which comprise at least two different antigen binding sites. Each of these antigen-binding sites will be capable of binding to a different epitope, which may be present on the same antigen or different antigens. The multi-specific antigen binding protein may have specificity for more than one antigen, for example two antigens, or for three antigens, or for four antigens.

Classification and formats of bispecific antibodies are comprehensively described in reviews by Labrijn et al 2019 and Brinkmann and Kontermann 2017. Bispecifics may be generally classified as having a symmetric or asymmetric architecture. Bispecifics may have an Fc or may be fragment-based (lacking an Fc). Fragment based bispecifics combine multiple antigen-binding antibody fragments in one molecule without an Fc region e.g. Fab-scFv, Fab-scFv2, orthoganol Fab-Fab, Fab-Fv, tandem scFc (e.g. BiTE and BIKE molecules), Diabody, DART, TandAb, scDiabody, tandem dAb etc.

In one aspect, the antibody is humanised or chimeric. In one embodiment, the recombinant polypeptide is an antibody, wherein the antibody is an IgG1, IgG4 or mAbdAb. As used herein, the term mAbdAb refers to a monoclonal antibody linked to a further binding domain, in particular a single variable domain such as a domain antibody. A mAbdAb has at least two antigen binding sites, at least one of which is from a domain antibody, and at least one is from a paired VH/VL domain. In a particular aspect, the antibody is a monoclonal antibody (mAb), such as, for example, an IgG1, or an IgG4. In an alternative aspect, the antibody is a bispecific antibody, for example a mAbdAb.

In another embodiment, the one or more impurities of the process are one or more of: host cell proteins (HCPs), nucleic acids, endotoxins, product variants, process variants, and/or cell culture media associated impurities. In an aspect, the one or more impurities are HCPs. In an aspect the nucleic acid is host cell DNA.

In one embodiment, the one or more impurities present in the process are produced by or derived from a host cell, which is a eukaryotic cell. In one aspect, the eukaryotic cell is a mammalian cell; a fungal cell; or a yeast cell. In one aspect, the one or more impurities are produced by or derived from a mammalian cell. In a further aspect, the mammalian cell is selected from: a human or rodent (such as a hamster or mouse) cell. In particular, the mammalian cell is selected from: CHO, NS0, Sp2/0, COS, K562, BHK, PER.C6, and/or HEK cells. In one aspect, the host cell is an HEK, CHO, PER.C6, Sp2/0, and/or NS0 cell. In another aspect, the yeast cell is Pichia pastoris, Saccharomyces cerevisiae, or Schizosaccharomyces pombe. In a further aspect, the fungal cell is Aspergillus sp. or Neurospora crassa.

In an alternative embodiment, the one or more impurities present in the process are produced by or derived from a host cell, which is a prokaryotic cell, for example a bacterial cell. In particular, the bacterial cell is: E. coli (for example, W3110, BL21); B. subtilis and/or other suitable bacteria.

In one embodiment the host cell protein is selected from: PLBL2 (Phospholipase B-Like 2 protein), cathepsin L, cathepsin D, thyrodoxin, neural cell adhesion molecule, renin receptor, lipoprotein lipase, chondroitin sulfate protoglycan 4, alpha-enolase, galectin-3-binding protein, G-protein coupled receptor 56, V-type proton ATPase subunit S1, Nidogen-1, ATP synthase subunit beta, mitochondrial, Vimentin, Heat shock protein, Actin, Peroxirodoxin 1, SPARC, Clusterin, Complement C1r-a sub-component, Metalloproteinase inhibitor 1, insulin, sulphated glycoprotein 1, and/or Lysosomal protective protein. In particular, the HCP is phospholipase B-Like 2 protein (PLBL2). PLBL2 has been found to be an HCP impurity that is difficult to remove during the downstream processing of antibodies due to its apparent binding to the recombinant polypeptide. In one aspect, the recombinant polypeptide is an antibody, such as an IgG antibody, in particular an IgG4 antibody. The PLBL2 amount can be measured using methods known in the art, such as by ELISA (enzyme-linked immunosorbent assay), for example the PLBL2-specific ELISA disclosed in WO2015/038884. In an alternative embodiment, the HCP is cathepsin L. Cathepsin L is a protease produced during CHO cell culture which can potentially degrade recombinant polypeptides that are antibodies. In one aspect, the recombinant polypeptide is an antibody, such as an IgG antibody, in particular an IgG1 antibody. In a further embodiment, the purification of the recombinant polypeptide from cathepsin L can be measured by a reduced cathepsin L activity (for example with PROMOKINE PK-CA577-K142, cathepsin L activity assay kit) in the eluate of step (c).

The amount of impurities, for example HCPs, present in the solution or eluate may be determined by ELISA, OCTET (assay system), or other suitable methods. In the Examples described herein, the HCP level is determined by ELISA. A reduction in HCP content may be shown when compared to a control wash step without saccharin, and/or when compared to, for example, clarified unprocessed bulk(CUB) (CCCF) prior to purification.

In one embodiment, the solution or eluate has an HCP content which is reduced by more than half of the HCP content in the initial load; for example the HCP content is reduced by 60% or more, 70% or more, 80% or more, or 90% or more.

In one embodiment, the solution or eluate has an HCP content which is =500 ppm, ≤100 ppm, ≤300 ppm, ≤250 ppm, ≤200 ppm, ≤150 ppm, ≤100 ppm, ≤75 ppm, or =50 ppm. In one aspect, the content of the impurity which is HCP is '200 ppm. In one aspect, the HCP content is ≤195 ppm, ≤190 ppm, ≤185 ppm, ≤180 ppm, ≤175 ppm, ≤170 ppm, ≤165 ppm, ≤160 ppm, ≤155 ppm, or ≤150 ppm. In one aspect, the HCP content is ≤190 ppm.

The amount of host cell nucleic acid, for example DNA, e.g. residual genomic DNA (rgDNA) can be determined by Polymerase Chain Reaction (PCR). In the Examples described herein, the rgDNA level is determined by qPCR and expressed as rg DNA pg/mg protein. A reduction in rgDNA content may be shown when compared to a control process without saccharin. In one embodiment, the solution or eluate has a rgDNA content which is reduced compared to the initial sample; for example the rgDNA content is reduced by 10 fold, 20 fold, 50 fold, 100 fold or more. In one embodiment the rgDNA is about 50,000 pg/mg or less, about 30,000 pg/mg or less, about 25,000 pg/mg or less, about 10,000 pg/mg or less, about 5,000 pg/mg or less, about 1,000 pg/mg or less, about 500 pg/mg or less, about 250 pg/mg or less, or about 100 pg/mg or less following the addition of saccharin.

The amount of PLBL2 can be determined by ELISA. In the Examples described herein, the PLBL2 level is determined by ELISA and expressed as PLBL2 ppm. A reduction in PLBL2 content may be shown when compared to a control process without saccharin. In one embodiment, the solution or eluate has a PLBL2 content which is reduced by more than half of the PLBL2content in the initial load; for example the PLBL2content is reduced by 60% or more, 70% or more, 80% or more, or 90% or more. In one embodiment the PLBL2 is about 50 ppm or less, about 25 ppm or less, about 20 ppm or less, about 15 ppm or less, about 10 ppm or less, or about 5 ppm or less following the addition of saccharin.

The monomer content of the purified recombinant polypeptide may be 80% or more, 85% or more, 90% or more, or 95% or more. Monomer is distinguished relative to the product-related impurities aggregates and fragments. The monomer purity of the purified recombinant polypeptide in the eluate is measured by SEC-HPLC in the Examples herein, alternative suitable methods may also be used. In one embodiment, the purified recombinant polypeptide in the eluate has a monomer content ranging from about 90% to about 100%. In one aspect, the purified recombinant polypeptide in the eluate has a monomer content of ≥90%, ≥94%, ≥95%, ≥96%, ≥97%, ≥98%, or ≥99%. In one aspect, the monomer content is ≥95%. In one aspect, the purified recombinant polypeptide in the eluate has a monomer content of ≥97%.

In an alternative aspect, the amount of aggregation of the purified recombinant polypeptide is <5% of the total purified polypeptide, for example <3%.

In one aspect, the purified recombinant polypeptide is an antibody.

The yield can be measured as the percentage of recombinant polypeptide resulting from the purification process as compared to the start of the process. It is known that purification methods can remove both impurities and the recombinant polypeptide, and so a balance must be struck. The yield of recombinant polypeptide may be 70% or more, 75% or more, 80% or more, 85% or more, or 90% or more.

In one embodiment, the purified recombinant polypeptide in the eluate has a monomer content of ≥95% and the eluate has an HCP content of ≤200 ppm. In one aspect, the purified recombinant polypeptide in the eluate has a monomer content of ≥97% and the eluate has an HCP content of ≤200 ppm. In a further embodiment, the HCP content is further reduced by subsequent downstream processing.

Often, purification of recombinant polypeptides from host cell proteins results in fragmentation of the recombinant polypeptide. The Applicant has discovered that when the purification methods described herein are utilized, the amount of recombinant polypeptide fragmentation is negligible. In one embodiment, the eluted recombinant polypeptide contains less than about 10%, about 9%, about 8%, about 7%, about 6%, about 5%, about 4%, about 3%, about 2%, about 1%, or about <1% fragmented recombinant polypeptide. For example, the recombinant polypeptide is an antibody and the eluted antibody contains less than about 10%, about 9%, about 8%, about 7%, about 6%, about 5%, about 4%, about 3%, about 2%, about 1%, or about <1% fragmented antibody. In a particular aspect, the purified recombinant polypeptide is less than about 2% fragmented. In one aspect, the purified recombinant polypeptide has about <1% fragmentation.

In one embodiment, a method for reducing the level of one or more impurities in a solution comprising a recombinant polypeptide and one or more impurities is provided, wherein the process is a purification process which comprises the addition of saccharin.

Herein provided is a process for reducing host cell proteins (HCP) from a solution comprising a recombinant polypeptide and one or more impurities, wherein the process is a purification process which comprises the addition of saccharin.

Herein provided is a process for reducing host cell DNA from a solution comprising a recombinant polypeptide and one or more impurities, wherein the process is a purification process which comprises the addition of saccharin.

Herein provided is a process for reducing PLBL2 from a solution comprising a recombinant polypeptide and one or more impurities, wherein the process is a purification process which comprises the addition of saccharin.

Herein provided is a process for increasing yield and reducing the level of one or more impurities from a solution comprising a recombinant polypeptide and one or more impurities, wherein the process is a purification process which comprises the addition of saccharin.

Herein provided is a process for increasing monomer content and reducing the level of one or more impurities from a solution comprising a recombinant polypeptide and one or more impurities, wherein the process is a purification process which comprises the addition of saccharin.

In one embodiment, there is provided a process for purifying a recombinant polypeptide from a solution comprising one or more Host Cell Proteins (HCPs),comprising:

(a) loading the solution of recombinant polypeptide and one or more HCPs onto a chromatography support which is Protein A;

(b) washing the chromatography support with a wash buffer comprising saccharin sodium salt hydrate; and

(c) eluting the recombinant polypeptide from the chromatography support with an elution buffer.

In one aspect, the recombinant polypeptide is an antibody.

In one aspect, the saccharin concentration is 0.1-1 M or about 1 M.

In one embodiment, the wash buffer used in the process comprises about 0.3 M to about 0.5 M or about 1 M saccharin sodium salt hydrate, 55 mM tris base, and 45 mM acetic acid. In another aspect, the wash buffer further comprises about 100 mM to about 250 mM of sodium caprylate, and about 300 mM to about 1 M of sodium acetate. In another aspect, the wash buffer further comprises 1.1 M arginine.

### DEFINITIONS

It is to be understood that the terminology used herein is for the purpose of describing particular embodiments only, and is not intended to be limiting. Unless defined otherwise, all technical and scientific terms used herein have the same meaning as commonly understood by one of ordinary skill in the art to which the invention pertains.

As used in this specification and the claims, the singular forms “a”, “an”, and “the” include plural referents unless the content clearly dictates otherwise. Thus, for example, reference to “a polypeptide” includes a combination of two or more polypeptides, and the like.

The word “comprise”, and variations such as “comprises” and “comprising”, will be understood to imply the inclusion of a stated integer or step or group of integers but not to the exclusion of any other integer or step or group of integers or steps. Thus, the term “comprising” encompasses “including” or “consisting” e.g. a process “comprising” X may consist exclusively of X or may include something additional e.g. X+Y. The term “consisting essentially of” limits the scope of the feature to the specified materials or steps and those that do not materially affect the basic characteristic(s) of the claimed feature. The term “consisting of” excludes the presence of any additional component(s).

“About” as used herein when referring to a measurable value such as an amount, a temporal duration, and the like, encompasses a suitable margin of error such as variations of ±20% or ±10%, including ±5%, ±1%, and ±0.1% from the specified value, as appropriate to perform the disclosed methods.

As used herein, “affinity chromatography” is a chromatographic method that makes use of the specific, reversible interactions between biomolecules rather than general properties of the biomolecule such as isoelectric point, hydrophobicity, or size, to effect chromatographic separation.

A “buffer” is a buffered solution that resists changes in pH by the action of its acid-base conjugate components. An “equilibration buffer” refers to a solution used to prepare the chromatography support for chromatography. A “loading buffer” refers to a solution used to load the solution of the recombinant polypeptide and impurities onto the support. The equilibration and loading buffers can be the same. The equilibration, load and wash buffers can be the same. A “wash buffer” refers to a solution used to remove impurities from the chromatography support after loading is completed. The “elution buffer” is used to remove the target recombinant polypeptide from the chromatography support.

A “salt” is a compound formed by the interaction of an acid and a base.

The “aliphatic carboxylate” can be either straight chained or branched. The aliphatic carboxylate can be an aliphatic carboxylic acid or salt thereof, or the source of the aliphatic carboxylate can be an aliphatic carboxylic acid or salt thereof. The aliphatic carboxylate is straight chained and selected from the group consisting of: methanoic (formic) acid, ethanoic (acetic) acid, propanoic (propionic) acid, butanoic (butyric) acid, pentanoic (valeric) acid, hexanoic (caproic) acid, heptanoic (enanthic) acid, octanoic (caprylic) acid, nonanoic (pelargonic) acid, decanoic (capric) acid, undecanoic (undecylic) acid, dodecanoic (lauric) acid, tridecanoic (tridecylic) acid, tetradecanoic (myristic) acid, pentadecanoic acid, hexadecanoic (palmitic) acid, heptadecanoic (margaric) acid, octadecanoic (stearic) acid, and icosanoic (arachididic) acid or any salts thereof. Accordingly, the aliphatic carboxylate can comprise a carbon backbone of 1-20 carbons in length. For example, an aliphatic carboxylate comprises a 6-12 carbon backbone. In another example, the aliphatic carboxylate is selected from the group consisting of: caproate, heptanoate, caprylate, decanoate, and dodecanoate. The source of the aliphatic carboxylate is selected from the group consisting of an aliphatic carboxylic acid, such as a sodium salt of an aliphatic carboxylic acid, a potassium salt of an aliphatic carboxylic acid, and an ammonium salt of an aliphatic carboxylic acid.

The “recombinant polypeptide comprising one or more impurities” may be a solution which is a cell culture medium, for example a cell culture feedstream. The feedstream may be filtered. The solution may be a Clarified Unprocessed Bulk (CUB) (or clarified cell culture harvest/supernatant/fermentation broth). The CUB is also known as a cell culture supernatant with any cells and/or cellular debris removed by clarification. The solution may be a lysed preparation of cells expressing the recombinant polypeptide (e.g. a lysate). Clarified Unprocessed Bulk (CUB) is equivalent to clarified cell culture fluid (CCCF), and both terms can be used interchangeably.

The term “impurity” refers to any product that does not share the same nature as the recombinant polypeptide of interest. In particular, impurity refers to any foreign or undesirable molecule that is present in the load sample prior to chromatography or after chromatography, in the eluate. There may be “process-related impurities” present. These are impurities that are present as a result of the process in which the polypeptide of interest is produced. For example, these include host cell proteins (HCPs), RNA, and DNA. “HCP” refers to proteins, not related to the polypeptide of interest, produced by the host cell during cell culture or fermentation, including intracellular and/or secreted proteins. An example of a host cell protein is a protease, which can cause damage to the recombinant polypeptide of interest if it is still present during and after purification. For example, if a protease remains in the sample comprising the polypeptide of interest, it can create “product-related” substances or impurities which were not originally present and are not desired. The presence of proteases can cause decay, e.g. fragmentation, of the polypeptide of interest over time during the purification process, and/or in the final formulation.

The term “impurities” as used herein also include components used to grow the cells or to ensure expression of the polypeptide of interest, for example, solvents (e.g. methanol used to culture yeast cells), antibiotics, methotrexate (MTX), media components, flocculants, etc. Also included are molecules that are part of the chromatography support that leach into the sample during, for example, Protein A, Protein G, or Protein L chromatography.

Impurities also include “product-related variants” which include proteins that retain their activity but are different in their structure, and proteins that have lost their activity because of their difference in structure. These product-related variants include, for example, high molecular weight species (HMWs), low molecular weight species (LMWs), aggregated proteins, prescursors, degraded proteins, misfolded proteins, underdisulfide-bonded proteins, fragments, and deamidated species.

The presence of any one of these impurities in the eluate can be measured to establish whether the wash step has been successful. For example, we have shown a reduction in the level of HCP, expressed as parts of HCP per million (ppm) of product (see the Examples). HCP detected in “ppm” is equivalent to ng/mg, whereas “ppb” (“parts per billion”) is equivalent to pg/mg. We have also shown a reduction in the level of DNA, expressed as residual genomic DNA (rgDNA) pg/mg (see the Examples). We have also shown a reduction in the level of PLBL2, expressed as parts per million (ppm) of product (see the Examples).

When used herein, the term “Protein A” encompasses Protein A recovered from a native source (e.g. the cell wall of Staphylococcus aureus), Protein A produced synthetically (e.g. by peptide synthesis or by recombinant techniques), and variants thereof which retain the ability to bind proteins which have a C_{H}2/C_{H}3 region. Protein A can also bind to the variable region of the heavy chain (VH3), the affinity of which is strengthened in the absence of an Fc region. Protein A can be purchased commercially, for example from Repligen or Pharmacia or GE Healthcare.

“Protein A affinity chromatography” or “Protein A chromatography” refers to a specific affinity chromatographic method that makes use of the affinity of the IgG binding domains of Protein A for the Fc portion and/or variable region of an immunoglobulin molecule. This Fc portion comprises human or animal immunoglobulin constant domains C_{H}2 and C_{H}3 or immunoglobulin domains substantially similar to these. In practice, Protein A chromatography involves using Protein A immobilized to a chromatography support which is a solid support. See Gagnon, Protein A Affinity Chromatography, Purification Tools for Monoclonal Antibodies, pp. 155-198, Validated Biosystems, (1996). Protein G and Protein L may also be used for affinity chromatography. Any suitable method can be used to affix the Protein A to the chromatography support. Methods for affixing proteins are well known in the art. See e.g. Ostrove, in Guide to Protein Purification, Methods in Enzymology, (1990) 182: 357-371. Such chromatography supports, with and without immobilized Protein A or Protein L, are readily available from many commercial sources such as Vector Laboratory (Burlingame, Calif.), Santa Cruz Biotechnology (Santa Cruz, Calif.), BioRad (Hercules, Calif.), Amersham Biosciences (part of GE Healthcare, Uppsala, Sweden) and Millipore (Billerica, Mass.).

The terms “polypeptide” and “protein” are interchangeable and refer to a polymer of amino acid residues and does not refer to a specific length of the product; thus, peptides, oligopeptides, and proteins are included within the definition of polypeptide. This term also does not refer to or exclude post-expression modifications of the polypeptide although chemical or post- expression modifications of these polypeptides may be included or excluded as specific embodiments. Therefore, for example, modifications to polypeptides that include the covalent attachment of glycosyl groups, acetyl groups, phosphate groups, lipid groups and the like are expressly encompassed by the term polypeptide. Further, polypeptides with these modifications may be specified as individual species to be included or excluded from the present disclosure. In one embodiment, the molecule is a polypeptide or their related analogs or derivatives thereof. A polypeptide can be of natural (tissue-derived) origins, recombinant or natural expression from prokaryotic or eukaryotic cellular preparations, or produced chemically via synthetic methods.

“Recombinant” when used with reference to a polypeptide indicates that the cell has been modified by the introduction of a heterologous nucleic acid or polypeptide or the alteration of a native nucleic acid or polypeptide.

The term “saccharin” encompasses its synonyms including: benzoic sulfimide; 2,3-dihydro-3-oxobenzisosulfonazole; o-sulfobenzimide; benzo[d]isothiazol-3(2H)-one 1,1-dioxide; and 2H-1λ6,2-benzothiazol-1,1,3-trione. The chemical structure of saccharin is depicted below.

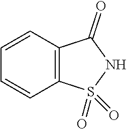 [SMILES: O=C1NS(=O)(=O)c2ccccc21]

References to “arginine” not only refer to the natural amino acids, but also encompass arginine derivatives or salts thereof, such as arginine HCl, acetyl arginine, agmatine, arginic acid, N-alpha-butyroyl-L-arginine, or N-alpha-pyvaloyl arginine.

The term “column volume” or (“CV”) refers to the total volume in a packed column.

The term “chromatography support” is interchangeable with “media”; “solid support”; “stationary phase” ; “resin”; “matrix”; “bead”; “gel”; or any other term that can be used to describe the material used to pack a chromatography column.

The abbreviation “MSS” refers to MABSELECT SURE resin, which is affinity chromatography media used for the capture of monoclonal antibodies (mAbs) at process scale.

The invention will now be described with the following examples.

### EXAMPLES

### Example 1: Protein A Column Chromatography Conditions

Protein A chromatography column was packed with MABSELECT SURE resin (GE Healthcare). Clarified Unprocessed Bulk (CUB) (CCCF) culture was from Chinese Hamster Ovary (CHO) cells expressing a recombinant polypeptide; a (i) bispecific antibody (mAb/dAb), (ii) monoclonal antibody 1 (mAb1), (iii) monoclonal antibody 2 (mAb2), (iv) monoclonal antibody 3 (mAb3), or (v) monoclonal antibody 4 (mAb4). 2-Sulfobenzoic Acid Ammonium Salt, Saccharin Sodium Salt Dihydrate, Saccharin Sodium Salt Hydrate and L-Arginine used were from Sigma Life Sciences. Where saccharin is mentioned in the examples, we mean saccharin sodium salt hydrate. Any other saccharin salts tested are further defined.

Instrumentation: AKTA AVANT preparative chromatography system used was from GE Healthcare.

### Chinese Hamster Ovary (CHO) Cell Culture for mAb1, mAb2, mAb3, mAb4 and mAb/dAb Production

Clarified Unprocessed Bulk (CUB) (CCCF) contained one of five antibody products: mAb/dAb (IgG1, MW=176kDa, pI=7.5); mAb1 (IgG1, MW=149kDa, pI=7.8); mAb2 (IgG1, MW=152 kDa, pI=9.6); mAb3 (IgG1, MW=147.6kDa, pI=7.8); or mAb4 (IgG4, MW=147.8kDa, pI=7.1). Similar methods were used to produce and harvest all products. For example, the mAb/dAb was prepared as follows: CHO K1A cells expressing the mAb/dAb were scaled up through a series of shake flasks to provide sufficient cells to inoculate a 50 L SARTORIUS Single Use Bioreactor (SUB). The 50 L

SUB was inoculated at a viable cell count of 1.0×10⁶ cell/mL and a working volume of 40 L. The culture was maintained at a fixed temperature; pH set point was also maintained until day 3 of the culture where it was reduced until the end of the cell culture batch. The culture, fed at regular points throughout the process, was harvested on day 14 using a 3M ZETA PLUS encapsulated filter system or Merck Millistak+ HC Pro encapsulated filter system.

### Protein A Chromatography

Protein A chromatography experiments were performed using all antibody products (mAb/dAb, mAb1, mAb2, mAb3 and mAb4) to determine the effect of different wash buffers on the final HCP content of the Protein A chromatography eluate (see Tables 1 and 2). Experiments were performed using an AKTA AVANT (GE Healthcare) and a Protein A chromatography column packed with MABSELECT SURE (GE Healthcare). The packing quality of the column was first assessed by measuring HETP (height equivalent to a theoretical plate) and peak asymmetry.

Apart from the saccharin containing washes, the post load Protein A washes tested are all variations of Protein A washes employed in the control Protein A process for antibody purification.

TABLE 1
Operating Conditions for Protein A Chromatography.

| Chromatography | | | |
|---|---|---|---|
| Step | | Composition | Volume |
| 1. | Equilibration | 55 mM tris base, 45 mM acetic acid, | 4CV |
| | | pH 7.5 or varied for Examples 8 (see | |
| | | Table 11) and 9 (see Table 12) | |
| 2. | Sample Load | CUB (CCCF) comprising mAb1, mAb2, | Varied |
| | | mAb3, mAb4 or mAb/dAb | |
| 3. | Wash | Varied (see Table 2) | 5CV |
| 4. | Equilibration | 55 mM tris base, 45 mM acetic acid, | 4CV or |
| | | pH 7.5 or varied for Examples 8 (see | 11CV for |
| | | Table 11) and 9 (see Table 12) | Examples |
| | | | 7-12 |
| 5. | Elution | 1.8 mM sodium acetate, 28.2 mM acetic | 5CV |
| | | acid, pH 3.6 | |
| 6. | Strip | 300 mM acetic acid | 3CV |
| 7. | Equilibration | 55 mM tris base, 45 mM acetic acid, | 1CV |
| | | pH 7.5 or varied for Examples 8 (see | |
| | | Table 11) and 9 (see Table 12) | |
| 8. | Cleaning | 0.1M sodium hydroxide | 3CV |
| 9. | Equilibration | 55 mM tris base, 45 mM acetic acid, | 3CV |
| | | pH 7.5 or varied for Examples 8 (see | |
| | | Table 11) and 9 (see Table 12) | |
| 10. | Storage | 20% Ethanol | 3CV |

The column volumes used are not intended to be limiting, for example: equilibration volumes can vary without affecting the process as long as enough equilibration buffer has passed though the column so that it is fully equilibrated (which can be measured by, for example, pH and conductivity of the column that of the equilibration buffer).

TABLE 2
Protein A wash buffers

| Wash Buffer Solution | Composition |
|---|---|
| Caprylate | 55 mM tris base, 45 mM acetic acid, 300 mM sodium acetate, |
| | 100 mM sodium caprylate, pH 7.5 |
| High Caprylate | 55 mM tris base, 45 mM acetic acid, 300 mM sodium acetate, |
| | 250 mM sodium caprylate, pH 7.5 |
| Caprylate High Acetate | 55 mM tris base, 45 mM acetic acid, 1.0M sodium acetate, |
| | 100 mM sodium caprylate, pH 7.5 |
| Caprylate + 0.3M Saccharin | 55 mM tris base, 45 mM acetic acid, 300 mM sodium acetate, |
| | 100 mM sodium caprylate, 300 mM saccharin sodium salt |
| | hydrate, pH 7.5 |
| Caprylate + 0.5M Saccharin | 55 mM tris base, 45 mM acetic acid, 300 mM sodium acetate, |
| | 100 mM sodium caprylate, 500 mM saccharin sodium salt |
| | hydrate, pH 7.5 |
| Caprylate + 1.1M L-Arginine | 55 mM tris base, 45 mM acetic acid, 300 mM sodium acetate, |
| | 100 mM sodium caprylate, 1.1M L-Arginine-HCl pH 7.5 (pH |
| | adjusted with tris base) |
| Equilibration Buffer | 55 mM tris base, 45 mM acetic acid, pH 7.5 |
| Equilibration buffer + 0.5M | 55 mM tris base, 45 mM acetic acid, 500 mM saccharin sodium |
| Saccharin | salt hydrate, pH 7.5 |
| No Caprylate + 0.1M Saccharin | 55 mM tris base, 45 mM acetic acid, 300 mM sodium acetate, |
| | 100 mM saccharin sodium salt hydrate, pH 7.5 |
| Equilibration buffer + 10 mM-3M | 55 mM tris base, 45 mM acetic acid, 10 mM-3M saccharin |
| Saccharin | sodium salt hydrate, pH 7.5 |
| Equilibration buffer + 0.3M | 55 mM tris base, 45 mM acetic acid, 300 mM saccharin sodium |
| Saccharin sodium salt dihydrate | salt dihydrate, pH 7.5 |
| Equilibration buffer + 1M | 55 mM tris base, 45 mM acetic acid, 1M saccharin sodium salt |
| Saccharin sodium salt dihydrate | dihydrate, pH 7.5 |
| PBS + 0.3M Saccharin sodium | PBS, 300 mM Saccharin sodium salt dihydrate |
| salt dihydrate | |
| PBS + 1M Saccharin sodium salt | PBS, 1M Saccharin sodium salt dihydrate |
| dihydrate | |
| MOPS pH 6.5 + 0.3M Saccharin | 100 mM MOPS in water, 300 mM saccharin sodium salt |
| sodium salt dihydrate | dihydrate, titrated to pH 6.5 |
| MOPS pH 6.5 + 1M Saccharin | 100 mM MOPS in water, 1M saccharin sodium salt dihydrate, |
| sodium salt dihydrate | titrated to pH 6.5 |
| MOPS pH 7.5 + 0.3M Saccharin | 100 mM MOPS in water, 300 mM saccharin sodium salt |
| sodium salt dihydrate | dihydrate, titrated to pH 7.5 |
| MOPS pH 7.5 + 1M Saccharin | 100 mM MOPS in water, 1M saccharin sodium salt dihydrate, |
| sodium salt dihydrate | titrated to pH 7.5 |
| MOPS pH 6.5 | 100 mM MOPS in water, titrated to pH 7.5 and pH 6.5 |
| MES pH 5.5 | 100 mM MES in water, titrated to pH 5.5 |
| MES pH 5.5 + 0.5M Saccharin | 100 mM MES in water, 500 mM saccharin sodium salt |
| sodium salt dihydrate | dihydrate, titrated to pH 5.5 |
| Buffer pH 5 | 25 mM Sodium Acetate, 12.1 mM Acetic Acid, pH 5.0 |
| Buffer pH 5 + 0.5M Saccharin | 25 mM Sodium Acetate, 12.1 mM Acetic Acid, 500 mM |
| sodium salt dihydrate | saccharin sodium salt dihydrate, pH 5.0 |
| Buffer pH 6 | 50.2 mM tris base, 49.8 mM acetic acid, pH 6 |
| Buffer pH 6 + 0.5M Saccharin | 50.2 mM tris base, 49.8 mM acetic acid, 500 mM saccharin |
| sodium salt dihydrate | sodium salt dihydrate, pH 6 |
| Buffer pH 7 | 50.2 mM tris base, 49.8 mM acetic acid, pH 7 |
| Buffer pH 7 + 0.5M Saccharin | 50.2 mM tris base, 49.8 mM acetic acid, 500 mM saccharin |
| sodium salt dihydrate | sodium salt dihydrate, pH 7 |
| Equilibration buffer + 0.5M | 55 mM tris base, 45 mM acetic acid, 500 mM saccharin sodium |
| Saccharin sodium salt dihydrate | salt dihydrate, pH 7.5 |
| Buffer pH 8 | 50.2 mM tris base, 49.8 mM acetic acid, pH 8 |
| Buffer pH 8 + 0.5M Saccharin | 50.2 mM tris base, 49.8 mM acetic acid, 500 mM saccharin |
| sodium salt dihydrate | sodium salt dihydrate, pH 8 |
| Equilibration buffer + 389.1 mM | 55 mM tris base, 45 mM acetic acid, 389.1 mM 2-sulfobenzoic |
| 2-sulfobenzoic acid ammonium | acid ammonium salt, pH 7.5 |
| salt | |
| Equilibration buffer + 389 mM | 55 mM tris base, 45 mM acetic acid, 389 mM saccharin sodium |
| saccharin sodium salt dihydrate | salt dihydrate, pH 7.5 |
| Equilibration buffer + 0.4M | 55 mM tris base, 45 mM acetic acid, 400 mM saccharin sodium |
| saccharin | salt hydrate, pH 7.5 |
| Equilibration buffer + 1.1M L- | 55 mM tris base, 45 mM acetic acid, 1.1M L-Arginine-HCl, |
| Arginine | titrated to pH 7.5 |
| Equilibration buffer + 100 mM | 55 mM tris base, 45 mM acetic acid, 100 mM sodium caprylate, |
| Caprylate | titrated to pH 7.5 |
| Equilibration buffer + 1.1M L- | 55 mM tris base, 45 mM acetic acid, 1.1M L-Arginine HC1, |
| Arginine + 0.5M Saccharin | 500 mM saccharin sodium salt dihydrate, titrated to pH 7.5 |
| sodium salt dihydrate | |
| Equilibration buffer + 100 mM | 55 mM tris base, 45 mM acetic acid, 100 mM sodium caprylate, |
| Caprylate + 0.5M Saccharin | 500 mM saccharin sodium salt dihydrate, titrated to pH 7.5 |
| sodium salt dihydrate | |
| Caprylate + 1.1M L-Arginine + | 55 mM tris base, 45 mM acetic acid, 300 mM sodium acetate, |
| 0.5M Saccharin sodium salt | 100 mM sodium caprylate, 1.1M L-Arginine-HCl, 500 mM |
| dihydrate | saccharin sodium salt dihydrate, titrated to pH 7.5 |
| Caprylate + 0.5M Saccharin | 55 mM tris base, 45 mM acetic acid, 300 mM sodium acetate, |
| sodium salt dihydrate | 100 mM sodium caprylate, 500 mM saccharin sodium salt |
| | dihydrate, titrated to pH 7.5 |

### Analysis: Host Cell Protein Concentration (HCP ELISA)

Host Cell Protein analysis using HCP ELISA was developed in-house to quantify the total amount of HCP in CHO-derived product samples (Mihara et al., (2015) J. Pharm. So. 104: 3991-3996). This HCP ELISA was developed using custom goat anti-CHO HCP polyclonal antibodies and an in-house produced HCP reference standard for multi-product use across CHO derived product.

### Analysis: PLBL2 Concentration (PLBL2 ELISA)

PLBL2 (Phospholipase B-Like 2) analysis using PLBL2 ELISA was developed in-house to quantify the total amount on PLBL2 in product samples. This PLBL2 ELISA was developed using custom mouse anti-PLBL2 monoclonal antibodies and an in-house produced PLBL2 reference standard.

### Analysis: Residual Genomic DNA (rgDNA)

DNA analysis was performed using an in-house developed qPCR method.

### Analysis: Purity Determination by Size Exclusion (SEC-HPLC)

The purity of the product (monomer) relative to recombinant polypeptide product related impurities (aggregates and fragments) was determined by Size Exclusion Chromatography using an SEC column (TOSOH TSKGEL G3000SWXL) on an AGILENT (1200 or 1260) HPLC system. Mobile phase, 100 mM sodium phosphate monobasic, 400 mM sodium chloride, pH 6.8; flow rate, 0.2 mL/min;

injection volume, 10 μL (5 mg/mL sample); detection at 280 nm (bandwidth of 8 nm).

### Example 2: Comparison of Three Different Washes on Protein A Using a mAb/dAb

The Protein A column was loaded to 28mgAb/mLResin using CUB (CCCF) from a CHO culture expressing a mAb/dAb. Using the Caprylate wash, HCP levels in the eluate were high given that the target HCP level in CHO derived drug products is generally <100 ppm. It is not critical to get below 100 ppm with the Protein A step, as some HCP removal can usually be gained from subsequent processing steps in an antibody purification process. Although, this can vary widely depending on the steps used and the strength/type of interaction between the product and specific HCPs. Increasing the Caprylate level in the Caprylate wash to 250 mM (High Caprylate) gets much closer to the target but at the expense of reducing monomer levels, meaning product has been lost and extra process steps may be required to remove the product related impurities. Addition of 0.3M sodium saccharin salt to the Caprylate wash (Caprylate+0.3M Saccharin) also gets close to the 100 ppm target at 178 ppm but, without sacrificing monomer.

TABLE 3
HCP and monomer levels in mAb/dAb Protein A eluates
where different washes have been applied

| Wash Buffer Solution | HCP (ppm) | % Monomer |
|---|---|---|
| Caprylate | 1007 | 99.3 |
| Caprylate High Caprylate | 123 | 93.9 |
| Caprylate + 0.3M Saccharin | 178 | 99.2 |

### Example 3: Comparison of 6 Wash Buffers on Protein A Using a mAb/dAb

The Protein A column was loaded to 35 mgAb/mLResin using CUB (CCCF) from a CHO culture expressing a mAb/dAb. Six different wash buffers were tested, including an equilibration buffer wash (containing no components to specifically remove HCPs) as seen in Table 4. As expected the HCP level in the resulting eluate was very high (4169 ppm). The concentration of sodium saccharin salt was increased in this example in an attempt to reduce HCP levels further. This gave good results when added to the Caprylate wash (59 ppm HCP) and when added to the equilibration buffer (135 ppm HCP). Increasing the sodium acetate level in the Caprylate wash was also tested here, but with little success (855 ppm HCP). Increasing the sodium caprylate level in the Caprylate wash again, gave relatively good HCP clearance but at the expense of monomer level.

TABLE 4
HCP and monomer levels in mAb/dAb Protein A eluates
where different washes have been applied

| Wash Buffer Solution | HCP (ppm) | % Monomer |
|---|---|---|
| Equilibration buffer | 4169 | 98.8 |
| Equilibration Buffer + 0.5M Saccharin | 135 | 99.0 |
| High Caprylate | 171 | 89.0 |
| Caprylate High Acetate | 855 | 98.9 |
| Caprylate + 0.5M Saccharin | 59 | 98.8 |
| Caprylate | 1214 | 98.7 |

### Example 4: Comparison of 7 Wash Buffers on Protein A Using mAb1

The Protein A column was loaded to 35 mgAb/mLResin using CUB (CCCF) from a CHO culture expressing mAb1. The same wash buffer solutions were tested here as in Example 1, with the addition of an arginine containing wash (see Table 5). The arginine containing wash resulted in a very low (79 ppm) HCP level. Saccharin (0.5M) was added to the Caprylate wash and gave the lowest HCP level (52 ppm HCP), and 0.5M saccharin added to the equilibration buffer also gave good results (135 ppm HCP). High Caprylate gave very low HCP but the monomer level was reduced from 95.9% to 53.5%. Increasing the sodium acetate level in the Caprylate wash from 300 mM to 1.0M (Caprylate High Acetate) gave no benefit in HCP removal over that achieved by the Caprylate wash for mAb1.

TABLE 5
HCP and monomer levels in mAb1 Protein A eluates
where different washes have been applied

| Wash Buffer Solution | HCP (ppm) | % Monomer |
|---|---|---|
| Equilibration Buffer | 2258 | 95.9 |
| Equilibration Buffer + 0.5M Saccharin | 135 | 96.3 |
| High Caprylate | 75 | 53.5 |
| Caprylate High Acetate | 981 | 95.9 |
| Caprylate + 0.5M Saccharin | 52 | 91.9 |
| Caprylate 1 | 987 | 95.9 |
| Caprylate + 1.1M L-Arginine | 79 | 96.0 |

### Example 5: Saccharin as CUB (CCCF) Additive to Reduce HCP Levels Post Protein A Chromatography

Saccharin was added, at different concentrations, to CUB (CCCF) from a CHO culture expressing a bispecific antibody (mAb/dAb). CHO cell culture production was as described in Example 1. The antibody was then purified by Protein A Chromatography. There were 6 Protein A runs in all, and for each, the Protein A column was loaded to a level of 31.4 mgAb/mLResin. For five of the chromatography runs, load was prepared by diluting CUB (CCCF) with sodium saccharin salt hydrate solution and water to give different concentrations of saccharin while maintaining the same antibody concentration (see Table 6). The sixth run was a control run comprising the control Protein A process; the CUB (CCCF) was loaded neat (no Saccharin or water added) and the Caprylate post load wash step was included. A summary of the chromatography conditions can be found in Tables 7 and 8.

The eluates from each of the 6 Protein A runs were analysed by HCP ELISA and SEC-HPLC as described in Example 1.

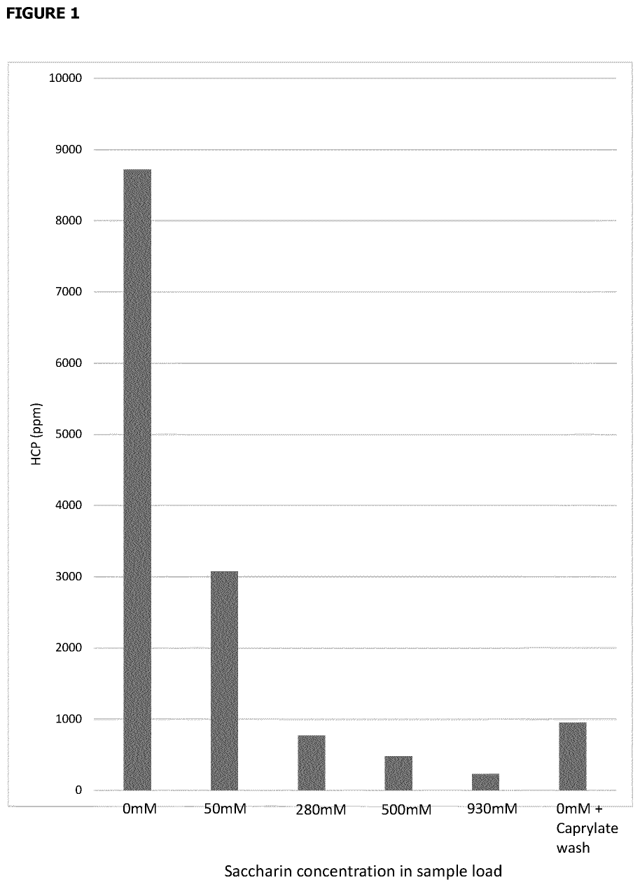

Results (see FIG. 1) show reducing levels of HCP present in Protein A eluate with increasing levels of sodium saccharin added to CUB (CCCF). 50 mM saccharin in the load resulted in a more than 50% decrease in HCP levels in the Protein A eluate compared to no saccharin in the load. 280 mM, 500mM and 630 mM saccharin in the load was superior at HCP clearance compared to Caprylate wash (run 6). The effect of reducing HCP levels increases as saccharin concentration is increased.

TABLE 6
Load preparation

| | CUB | Saccharin | Water | Final | Final Ab |
|---|---|---|---|---|---|
| | (CCCF) vol. | solution vol. | vol. | Saccharin | Conc. |
| Run | (mL) | (mL) | (mL) | conc. (mM) | (mg/mL) |
| 1 | 130 | 65 | 0 | 930 | 0.95 |
| 2 | 130 | 20 | 45 | 280 | 0.95 |
| 3 | 130 | 3.5 | 61.5 | 50 | 0.95 |
| 4 | 130 | 0 | 65 | 0 | 0.95 |
| 5 | 130 | 35 | 30 | 500 | 0.95 |
| 6 | No CUB | n/a | n/a | n/a | 1.43 |
| | (CCCF) | | | | |
| | dilution | | | | |

TABLE 7
Summary Protein A Load and Wash conditions

| | Load | Load | Load | |
|---|---|---|---|---|
| | Saccharin | mass | vol | |
| Run | Conc. (mM) | (mg) | (mL) | Post load wash |
| 1 | 930 | 114 | 120 | 55 mM tris base, 45 mM acetic acid, |
| | | | | pH 7.5 |
| 2 | 280 | 114 | 120 | 55 mM tris base, 45 mM acetic acid, |
| | | | | pH 7.5 |
| 3 | 50 | 114 | 120 | 55 mM tris base, 45 mM acetic acid, |
| | | | | pH 7.5 |
| 4 | 0 | 114 | 120 | 55 mM tris base, 45 mM acetic acid, |
| | | | | pH 7.5 |
| 5 | 500 | 114 | 120 | 55 mM tris base, 45 mM acetic acid, |
| | | | | pH 7.5 |
| 6 | 0 | 114 | 79.7 | 55 mM tris base, 45 mM acetic acid, |
| | | | | 300 mM sodium acetate, 100 mM |
| | | | | sodium caprylate, pH 7.5 |

TABLE 8
Operating conditions for Protein A Chromatography

| Chromatography | | | |
|---|---|---|---|
| Step | | Composition | Volume |
| 1. | Equilibration | 55 mM tris base, 45 mM acetic acid, | 4CV |
| | | pH 7.5 | |
| 2. | Sample Load | mAb/dAb CUB (CCCF) with Saccharin | Varied |
| | | (see Table 6) | |
| 3. | Wash | Varied (See Table 7) | 5CV |
| 4. | Equilibration | Equilibration: 55 mM tris base, 45 mM | 4CV |
| | | acetic acid, pH 7.5 | |
| 5. | Elution | 1.8 mM sodium acetate, 28.2 mM acetic | 5CV |
| | | acid, pH 3.6 | |
| 6. | Strip | 300 mM acetic acid | 3CV |
| 7. | Equilibration | 55 mM tris base, 45 mM acetic acid, | 1CV |
| | | pH 7.5 | |
| 8. | Cleaning | 0.1M sodium hydroxide | 3CV |
| 9. | Equilibration | 55 mM tris base, 45 mM acetic acid, | 3CV |
| | | pH 7.5 | |
| 10. | Storage | 20% Ethanol | 3CV |

### Example 6: MABSELECT SURE Protein A Column Wash Screening for mAb2

A sodium saccharin wash was tested on Protein A for mAb2 and compared to an Arginine wash.

### Methods and Materials

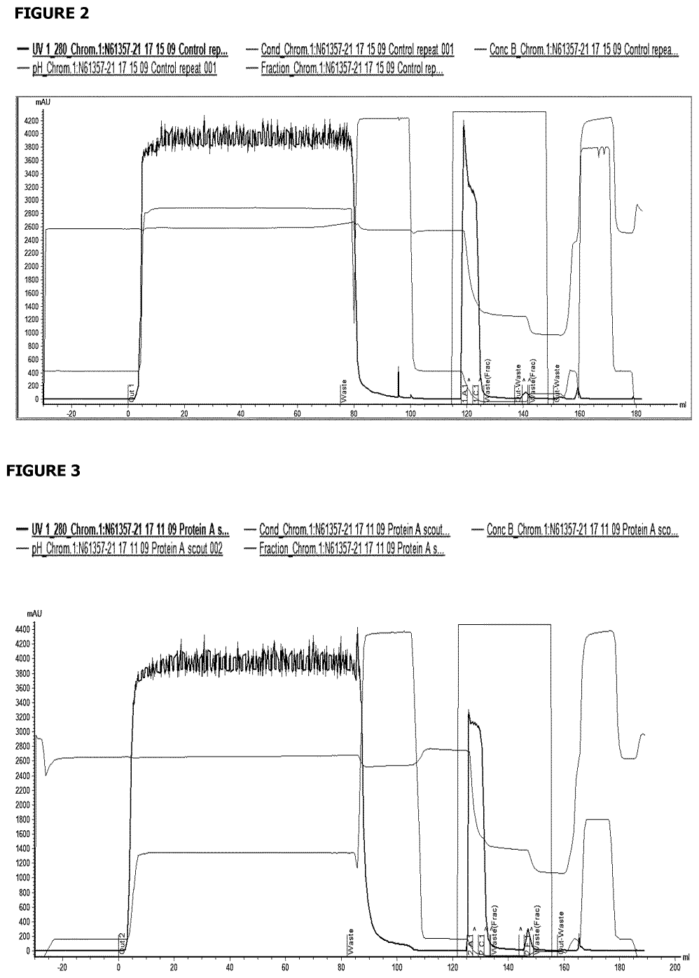

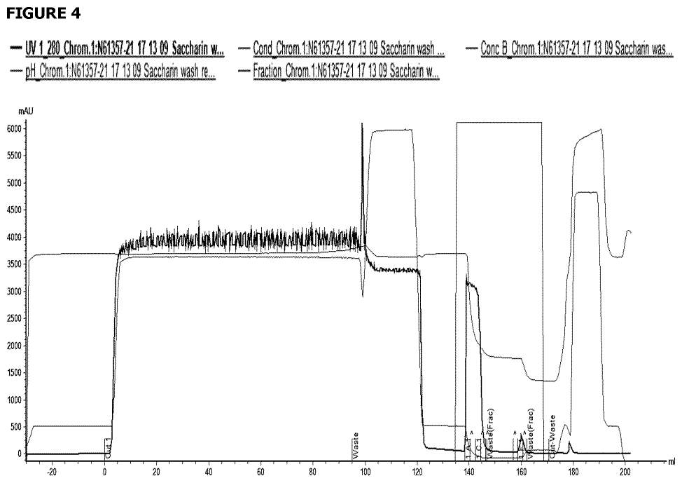

An antibody mAb2 was filtered using 0.2 μM STERICUP filters, and used in the following runs. Small scale screening experiments were performed:
Caprylate wash;
Caprylate+1.1M L-Arginine (buffer containing 1.1 M L-Arginine in the Caprylate wash);
Equilibration buffer+0.5M Saccharin (buffer containing 500 mM Saccharin in the Caprylate equilibration buffer).
   Table 9 shows the recovery, eluate monomer purity and HCP data of the three different Protein A runs (Caprylate, Caprylate+Arginine, and Caprylate+Saccharin as shown in FIGS. 2, 3, and 4).

TABLE 9
Summary of results of antibody mAb2 purification
from Protein A Column Wash Screening

| | Recovery | Monomer | HCP level |
|---|---|---|---|
| Experiment Name | (%) | Purity (%) | (ppm) |
| Caprylate | 101.28 | 93.6 | 4517.87 |
| Caprylate + 1.1M L- | 86.82 | 96.1 | 357.20 |
| Arginine | | | |
| Equlibration buffer + 0.5M | 89.57 | 97.3 | 352.20 |
| Saccharin | | | |

Saccharin was added to Protein A equilibration buffer, whereas in the arginine wash, arginine was added to the Caprylate Protein A wash buffer which also contained sodium acetate and sodium caprylate. According to the data in this example, the use of saccharin in the wash buffer leads to the highest monomer purity (97.3%), whilst also maintaining a high recovery and a low HCP level.
FIGS. 2, 3, and 4 show the MSS chromatograms for the caprylate, arginine and saccharin wash runs of the antibody mAb2 respectively.

### Example 7: Comparison of 12 Wash Buffers on Protein A Using mAb3

The Protein A column was loaded to 35 mgAb/mLresin using CUB (CCCF) from a CHO culture expressing mAb3. Protein A wash buffers with different saccharin concentrations ranging from 10 mM-3M saccharin sodium salt hydrate (Equilibration buffer+10mM-3M Saccharin in Table 2) were tested.

The results show (see Table 10) that 10 mM saccharin was effective for HCP removal, and increasing the saccharin concentration improved HCP clearance. The Equilibration buffer negative control observed HCP levels of 3008 ppm which was reduced by over 100-fold with 3M saccharin in the wash step. The monomer purity was comparable across the different eluates, up until 1.5M saccharin. HCP clearance for 200 mM saccharin was comparable to the Caprylate wash buffer and 600 mM saccharin was comparable to the High Caprylate wash buffer.

TABLE 10
HCP, monomer and yield levels in mAb3 Protein A eluates where washes
with different saccharin concentrations have been applied.

| | HCP | | |
|---|---|---|---|
| Wash Buffer Solution | (ppm) | % Monomer | % Yield |
| High Caprylate | 85.42 | 84.836 | 70.55 |
| Caprylate | 311.05 | 98.964 | 90.91 |
| No Caprylate + 0.1M Saccharin | 601.67 | 98.926 | 90.65 |
| Equilibration buffer | 3008.43 | 98.929 | 68.69 |
| Equilibration buffer + 10 mM saccharin | 2369.68 | 98.696 | 91.69 |
| Equilibration buffer + 0.1M saccharin | 2103.31 | 98.726 | 88.51 |
| Equilibration buffer + 0.2M saccharin | 424.51 | 98.797 | 91.89 |
| Equilibration buffer + 0.4M saccharin | 217.21 | 98.953 | 94.28 |
| Equilibration buffer + 0.6M saccharin | 114.91 | 98.932 | 75.21 |
| Equilibration buffer + 0.8M saccharin | 61.49 | 98.837 | 85.25 |
| Equilibration buffer + 1.5M saccharin | 35.32 | 40.963 | 42.33 |
| Equilibration buffer + 3M saccharin | 29.52 | 18.67 | 12.58 |

### Example 8: Comparison of 13 Wash Buffers on Protein A Using mAb3

The Protein A column was loaded to 35 mgAb/mLresin using CUB (CCCF) from a CHO culture expressing mAb3. Protein A wash buffers with different buffering systems (Equilibration buffer, PBS, MOPS pH 7.5, MOPS pH 6.5, and MES pH 5.5) and with (0.3M, 0.5M or 1M) or without saccharin sodium salt dihydrate were tested.

The use of saccharin sodium salt dihydrate at 0.3M, 0.5M and 1M in the wash buffer consistently reduced HCP levels across all buffering systems tested (see Table 11). The DNA results show that saccharin sodium salt dihydrate provided good DNA clearance at 1M concentration compared to the Caprylate wash.

TABLE 11
HCP, rgDNA and monomer levels in mAb3 Protein A eluates where
washes with different buffering systems have been applied

| Equilibration | | HCP | rgDNA | Monomer |
|---|---|---|---|---|
| Buffer Solution | Wash Buffer Solution | (ppm) | (pg/mg) | (%) |
| Equilibration | Equilibration buffer + 0.3M Saccharin | 491 | 317820 | 98.8 |
| buffer | sodium salt dihydrate | | | |
| Equilibration | Equilibration buffer + 1M Saccharin | 60 | 88 | 98.6 |
| buffer | sodium salt dihydrate | | | |
| PBS | PBS + 0.3M Saccharin sodium salt | 263 | 6283 | 99.0 |
| | dihydrate | | | |
| PBS | PBS + 1M Saccharin sodium salt | 47 | 548 | 99.0 |
| | dihydrate | | | |
| MOPS pH 7.5 | MOPS pH 7.5 + 0.3M Saccharin sodium | 700 | 30249 | 98.9 |
| | salt dihydrate | | | |
| MOPS pH 7.5 | MOPS pH 7.5 + 1M Saccharin sodium | 63 | 130 | 99.0 |
| | salt dihydrate | | | |
| MOPS pH 6.5 | MOPS pH 6.5 + 0.3M Saccharin sodium | 160 | 823 | 99.1 |
| | salt dihydrate | | | |
| MOPS pH 6.5 | MOPS pH6.5 + 1M Saccharin sodium | 45 | 81 | 99.1 |
| | salt dihydrate | | | |
| Equilibration | Caprylate | 570 | 20949 | 99.1 |
| buffer | | | | |
| Equilibration | Equilibration buffer | 3776 | | 98.4 |
| buffer | | | | |
| MOPS pH 6.5 | MOPS pH 6.5 | 3222 | | 98.5 |
| MES pH 5.5 | MES pH 5.5 | 2335 | | 98.5 |
| MES pH 5.5 | MES pH 5.5 + 0.5M Saccharin sodium | 215 | | 98.6 |
| | salt dihydrate | | | |

### Example 9: Comparison of 14 Wash Buffers on Protein A Using mAb3

The Protein A column was loaded to 35 mgAb/mLresin using CUB (CCCF) from a CHO culture expressing mAb3. Protein A wash buffers with different pH's ranging from pH 5 to pH 8 and with (0.3M or 0.5M) or without saccharin sodium salt dihydrate were tested. Table 12 shows that the use of saccharin sodium salt dihydrate at all pH's tested reduced HCP levels. The monomer levels were comparable across all pH's.

TABLE 12
HCP and monomer levels in mAb3 Protein A eluates
where washes with different pH's have been applied

| Equilibration | | Monomer | HCP ppm | % decrease |
|---|---|---|---|---|
| Buffer Solution | Wash Buffer Solution | (%) | (ng/mg) | in HCP |
| Buffer pH 5 | Buffer pH 5 | 98.5 | 2041 | 94.3 |
| Buffer pH 5 | Buffer pH 5 + 0.5M Saccharin | 98.7 | 116 | |
| | sodium salt dihydrate | | | |
| MES pH 5.5 | MES pH 5.5 | 98.5 | 2335 | |
| MES pH 5.5 | MES pH 5.5 + 0.5M Saccharin | 98.6 | 215 | 90.8 |
| | sodium salt dihydrate | | | |
| Buffer pH 6 | Buffer pH 6 | 98.5 | 1691 | 79.8 |
| Buffer pH 6 | Buffer pH 6 + 0.5M Saccharin | 98.6 | 342 | |
| | sodium salt dihydrate | | | |
| MOPS pH 6.5 | MOPS pH 6.5 | 98.5 | 3222 | 95.0 |
| MOPS pH 6.5 | MOPS pH 6.5 + 0.3M Saccharin | 99.1 | 160 | |
| | sodium salt dihydrate | | | |
| Buffer pH 7 | Buffer pH 7 | 98.4 | 2247 | 93.2 |
| Buffer pH 7 | Buffer pH 7 + 0.5M Saccharin | 98.5 | 153 | |
| | sodium salt dihydrate | | | |
| Equilibration | Equilibration buffer | 98.4 | 3776 | 94.3 |
| Buffer | | | | |
| Equilibration | Equilibration buffer + 0.5M | 98.5 | 213 | |
| Buffer | Saccharin sodium salt dihydrate | | | |
| Buffer pH 8 | Buffer pH 8 | 98.5 | 3285 | 53.4 |
| Buffer pH 8 | Buffer pH 8 + 0.5M Saccharin | 97.8 | 1530 | |
| | sodium salt dihydrate | | | |

### Example 10: Comparison of 5 Wash Buffers on Protein A Using mAb3

The Protein A column was loaded to 35 mgAb/mLresin using CUB (CCCF) from a CHO culture expressing mAb3. Protein A wash buffers with different saccharin salts were tested. All three salt variants of saccharin decreased HCP levels (see Table 13). Saccharin and saccharin sodium salt dihydrate in the wash buffer resulted in a >90% decrease in HCP levels compared to the Equilibration buffer. The saccharin salts did not have an impact on monomer levels.

TABLE 13
HCP and monomer levels in mAb3 Protein A eluates where washes
with different saccharin salt variants have been applied

| | | | % decrease |
|---|---|---|---|
| | | | in HCP from |
| | Monomer | HCP | Equilibration |
| Wash Buffer Solution | (%) | (ppm) | buffer |
| Equilibration buffer + 389.1 mM | 97.9 | 615 | 83.7 |
| 2-sulfobenzoic acid ammonium salt | | | |
| Equilibration buffer + 389 mM | 98.0 | 240 | 93.6 |
| saccharin sodium salt dihydrate | | | |
| Equilibration buffer + 0.4M | 99.0 | 217 | 94.2 |
| saccharin | | | |
| Caprylate | 97.7 | 598 | 84.1 |
| Equilibration buffer | 97.6 | 3768 | |

### Example 11: Comparison of 4 Wash Buffers on Protein A Using mAb4

mAb4 is an IgG4 antibody that is known to co-purify with high levels of PLBL2. The Protein A column was loaded to 35 mgAb/mLresin using CUB (CCCF) from a CHO culture expressing mAb4. The Protein A wash buffers Equilibration buffer+0.5M Saccharin sodium salt dihydrate and Caprylate+1.1M L-Arginine were tested and compared. The Caprylate+1.1M L-Arginine wash buffer has previously been implemented to reduce PLBL2 levels in the protein A purification of mAb4 and was used as a positive control. Saccharin sodium salt dihydrate in the wash buffer was superior in reducing PLBL2 levels in the protein A purification of mAb4 compared to arginine in the wash buffer (see Table 14).

TABLE 14
PLBL2 and monomer levels in mAb4 Protein A eluates
where a wash with saccharin has been applied

| Wash Buffer Solution | PLBL2 (ppm) | Monomer (%) |
|---|---|---|
| Caprylate | 320 | 98.3 |
| Equilibration buffer + 0.5M Saccharin | 1.8 | 98.4 |
| sodium salt dihydrate | | |
| Equilibration buffer | 293 | 98.3 |
| Caprylate + 1.1M L-Arginine | 3.7 | 98.3 |

### Example 12: Comparison of 6 Wash Buffers on Protein A Using mAb3

The Protein A column was loaded to 35 mgAb/mLresin using CUB (CCCF) from a CHO culture expressing mAb3. Protein A wash buffers with saccharin sodium salt dihydrate plus other buffer components were tested to investigate possible synergistic effects in HCP clearance. Table 15 demonstrates that Equilibration buffer+0.5M Saccharin sodium salt dihydrate reduced HCP levels by 89.2%, Equilibration buffer+1.1M L-Arginine reduced HCP levels by 87.2% and Equilibration buffer+100 mM Caprylate reduced HCP levels by 70.5% compared to the Equilibration buffer control.

Therefore, saccharin sodium salt dihydrate in the wash buffer was superior in reducing HCP levels for mAb3.

The addition of saccharin sodium salt dihydrate to Equilibration buffer+1.1M L-Arginine and Equilibration buffer+100 mM Caprylate reduced HCP levels by 98.1% and 98.7%, respectively, indicating increased HCP clearance when saccharin sodium salt dihydrate is combined with other buffer components.

Table 16 shows a similar synergistic effect when saccharin sodium salt dihydrate was added to the Caprylate wash (96.7% reduction of HCP levels) and when saccharin sodium salt dihydrate was added to Caprylate+1.1M L-Arginine (96.9% reduction of HCP levels).

TABLE 15
HCP levels in mAb3 Protein A eluates where washes with a combination
of saccharin and other buffer components have been applied

| | | % decrease |
|---|---|---|
| | | in HCP from |
| | | Equilibration |
| Wash Buffer Solution | HCP (ppm) | buffer |
| Equilibration buffer + 0.5M Saccharin | 212.87 | 89.2 |
| sodium salt dihydrate | | |
| Equilibration buffer + 1.1M L-Arginine | 253.19 | 87.2 |
| Equilibration buffer + 100 mM Caprylate | 582.78 | 70.5 |
| Equilibration buffer + 1.1M L-Arginine + | 38.01 | 98.1 |
| 0.5M Saccharin sodium salt dihydrate | | |
| Equilibration buffer | 1975.39 | — |
| Equilibration buffer + 100 mM Caprylate + | 26.76 | 98.7 |
| 0.5M Saccharin sodium salt dihydrate | | |

TABLE 16
HCP levels in mAb3 Protein A eluates where washes with a combination
of saccharin and other buffer components have been applied

| | | % decrease |
|---|---|---|
| | | in HCP from |
| | | Equilibration |
| Wash Buffer Solution | HCP (ppm) | buffer |
| Caprylate + 1.1M L-Arginine | 177.16 | 95.3 |
| Caprylate + 1.1M L-Arginine + 0.5M | 115.44 | 96.9 |
| Saccharin sodium salt dihydrate | | |
| Caprylate | 496.26 | 86.9 |
| Caprylate + 0.5M Saccharin sodium salt | 125.74 | 96.7 |
| dihydrate | | |
| Equilibration buffer | 3775.76 | 0 |
| Equilibration buffer + 0.5M Saccharin | 212.87 | 94.3 |
| sodium salt dihydrate | | |

1. A process for purifying a recombinant polypeptide from a solution comprising one or more impurities, wherein the process is a chromatography process which comprises the addition of saccharin, wherein the saccharin concentration is from about 0.01 M to about 4 M, and wherein the saccharin is not used to displace the recombinant polypeptide from a chromatography support.

2. The process according to claim 1, wherein the chromatography process comprises at least one of: affinity chromatography; ion exchange chromatography; anion exchange chromatography; cation exchange chromatography; gel-permeation or gel filtration chromatography; dye-ligand chromatography; hydrophobic interaction chromatography (HIC); mixed mode chromatography (MMC); and ceramic hydroxyapatite chromatography.

3. The process according to claim 1, wherein the process comprises at least one of: (a) a loading step; and, (b) a washing step.

4. The process according to claim 3, wherein the process comprises one or more chromatography steps (a), (b):
(a) a loading step comprising a loading solution comprising the recombinant polypeptide and one or more impurities, wherein loading buffer is applied onto a chromatography support;
(b) a washing step comprising a washing buffer wherein the washing buffer is applied onto the chromatography support; and further comprises (c):
(c) an elution step comprising an elution buffer wherein the elution buffer is applied onto the chromatography support.

5. The process according to claim 1 wherein the chromatography process is Protein A affinity chromatography.

6. The process according to claim 1 wherein the solution comprising the recombinant polypeptide and one or more impurities, is a cell culture feedstream, or a clarified cell culture fluid.

7. The process according to claim 1 wherein the recombinant polypeptide is an antigen binding protein.

8. The process according to claim 7 wherein the antigen binding protein is an antibody.

9. The process according to claim 1 wherein the one or more impurities are derived from a mammalian cell.

10. The process according to claim 9 wherein the mammalian cell is selected from the group consisting of: CHO (Chinese Hamster Ovary); NS0; Sp2/0; COS; K562; BHK; PER.C6; and, HEK cells.

11. The process according to claim 1 wherein the one or more impurities in the process are one or more of: host cell proteins (HCPs), nucleic acids, endotoxins, and cell culture media associated impurities.

12. The process according to claim 1 wherein the saccharin concentration is from about 0.01 M to about 3.0 M; from about 0.05 M to about 1.0 M; or from about 0.3 M to about 1.5 M.

13. The process according to claim 4 wherein one or more of the solution, a loading buffer, a washing buffer, and an elution buffer; any one or more of which further comprises one or more of arginine; caprylate; lysine; and, sodium acetate.

14. The process according to claim 1 wherein saccharin is added to one or more of the solution, loading buffer, and washing buffer.

15. The process according to claim 14 wherein saccharin is in the form of 2-sulfobenzoic acid ammonium salt; saccharin sodium salt dihydrate; or saccharin sodium salt hydrate.

16. The process according to claim 1 wherein the purified recombinant polypeptide is further purified.

17. The process according to claim 1 wherein the purified recombinant polypeptide is formulated to a final therapeutic formulation.

18. A wash or load buffer for purifying using chromatography a recombinant polypeptide from a solution comprising one or more impurities, wherein the wash or load buffer comprises saccharin, and wherein the saccharin concentration is from about 0.01 M to about 4 M.

19. A cell culture feedstream comprising a recombinant polypeptide and one or more impurities, wherein the feedstream is a solution comprising saccharin, and wherein the saccharin concentration is from about 0.01 M to about 4 M.

20. (canceled)

21. A process for purifying an antibody from a solution comprising the antibody and one or more impurities derived from a mammalian cell, the process comprising a chromatography process comprising a chromatography support and:
(a) loading the solution comprising the antibody and one or more impurities derived from a mammalian cell on to the chromatography support by applying the solution and a loading buffer to the chromatography support;
(b) washing the chromatography support by applying a washing buffer to the chromatography support;
(c) eluting the antibody from the chromatography support by applying an elution buffer to the chromatography support;
wherein one or more of the loading buffer and the washing buffer comprise from about 0.3 M to about 1.5 M saccharin.

22. The process of claim 21 wherein:
(1) the chromatography process is Protein A chromatography; and,
(2) one or more of the solution, the loading buffer, the washing buffer, and the elution buffer; further comprises one or more of arginine, caprylate, lysine, and, sodium acetate.
